(19) United States
(12) Patent Application Publication
(10) Pub. No.: US 2026/0363631 A1
(43) Pub. Date: Nov. 19, 2026

(54) INFORMATION PROCESSING APPARATUS, METHOD, AND PROGRAM

(71) Applicant: FUJIFILM Corporation, Tokyo (JP)

(72) Inventor: Takuya YUZAWA, Tokyo (JP)

(73) Assignee: FUJIFILM Corporation, Tokyo (JP)

(21) Appl. No.: 19/667,682

(22) Filed: May 5, 2026

(30) Foreign Application Priority Data
May 13, 2025 (JP) 2025-080719

(51) Int. Cl. A61B 5/00; G06T 7/00; G06T 7/62; G06T 11/60; G06V 10/25; G06V 20/50; G06V 20/70

(52) U.S. Cl. CPC A61B 5/743; G06T 7/0012; G06T 7/62; G06T 11/60; G06V 10/25; G06V 20/50; G06V 20/70; G06T 2207/30096; G06T 2210/41; G06V 2201/031

(57) ABSTRACT

A processor acquires a measurement result of an object included in a medical image, recognizes an organ region including the object in the medical image, derives a score representing suitability for displaying the measurement result based on a ratio at which a measurement label, which includes the measurement result, and the organ region overlap each other, for each of a plurality of candidate regions for displaying the measurement result around the object, and displays the measurement result in at least one candidate region in which the score satisfies a criterion.

### CROSS REFERENCE TO RELATED APPLICATIONS

The present application claims priority from Japanese Patent Application No. 2025-080719, filed on May 13, 2025, the entire disclosure of which is incorporated herein by reference.

### BACKGROUND

### Technical Field

The present disclosure relates to an information processing apparatus, an information processing method, and an information processing program.

### Related Art

In the related art, an object such as an organ or a lesion included in a medical image such as a computed tomography (CT) image, a magnetic resonance imaging (MRI) image, an ultrasound image, or a radiation image is extracted, a size, a signal value, and the like of the object are measured, a measurement result is displayed in the medical image, and the presence or absence of an abnormality in the organ, a degree of growth of the lesion, and the like are checked with reference to the displayed measurement result.

In a case of displaying the measurement result, a method has been proposed in which a relationship between the object and the measurement result can be understood by displaying the measurement result in the vicinity of the object or connecting the object and the measurement result with a line or an arrow (for example, see JP2016-049327A). In addition, a method has been proposed in which a leader line connecting the object and the measurement result does not overlap a region in which the measurement result is displayed (see JP2009-232982A).

In the methods described in JP2016-049327A and JP2009-232982A, the measurement result may overlap the organ or the object on which the measurement is performed. In this case, the measurement result interferes with image interpretation. A position at which the measurement result is displayed can be moved by drag and drop, but such work is extremely troublesome for the operator.

### SUMMARY OF THE INVENTION

The present disclosure has been made in view of the above circumstances, and an object of the present disclosure is to enable a measurement result of an object included in a medical image to be displayed at a position that does not interfere with image interpretation.

An information processing apparatus according to the aspect of the present disclosure comprises:
a processor,
in which the processor is configured to:
   acquire a measurement result of an object included in a medical image;
   recognize an organ region including the object in the medical image;
   derive a score representing suitability for displaying the measurement result based on a ratio at which a measurement label, which includes the measurement result, and the organ region overlap each other, for each of a plurality of candidate regions for displaying the measurement result around the object; and
   display the measurement result in at least one candidate region in which the score satisfies a criterion.

In the information processing apparatus according to the aspect of the present disclosure, the processor may be configured to:
derive the ratio for each of the plurality of candidate regions by disposing the measurement label in the plurality of candidate regions; and
derive the score for each of the plurality of candidate regions based on the ratio.

In the information processing apparatus according to the aspect of the present disclosure, the processor may be configured to:
display a measurement object including at least two control points for measurement on the object of the medical image;
acquire the measurement result by receiving an operation on the control point of the measurement object;
set the plurality of candidate regions based on each of the at least two control points of the measurement object; and
reduce the score in a case where the measurement label disposed in each of a plurality of candidate regions based on one control point of the at least two control points (i) overlaps with another control point other than the one control point, (ii) overlaps with the measurement label disposed in each of the plurality of candidate regions based on the other control point, or (iii) overlaps with the measurement object including the one control point.

In the information processing apparatus according to the aspect of the present disclosure, the processor may be configured to reduce the score in a case where each of the plurality of candidate regions for one object overlaps with another object in a case of displaying the measurement result of a plurality of the objects.

In the information processing apparatus according to the aspect of the present disclosure, the processor may be configured to:
display a measurement object including at least two control points for measurement on the object of the medical image;
acquire the measurement result by receiving an operation on the control point of the measurement object;
set the plurality of candidate regions based on each of the at least two control points of the measurement object;
reduce the score in a case where the measurement label disposed in each of a plurality of candidate regions based on one control point of the at least two control points (i) overlaps with another control point other than the one control point, (ii) overlaps with the measurement label disposed in each of the plurality of candidate regions based on the other control point, or (iii) overlaps with the measurement object including the one control point;
further reduce the score (iv) in a case where each of the plurality of candidate regions for one object overlaps with another object in a case of displaying the measurement result of a plurality of the objects; and
increase a degree of the score reduction in a case of (iii) and (iv) than in a case of (i) and (ii).

In the information processing apparatus according to the aspect of the present disclosure, the plurality of candidate regions may include four regions of upper right, lower right, lower left, and upper left of at least two control points included in a measurement object for acquiring the measurement result.

In the information processing apparatus according to the aspect of the present disclosure, the processor may be configured to display the measurement result in a mode different from a mode of the candidate region for displaying the measurement result.

In the information processing apparatus according to the aspect of the present disclosure, the processor may be configured to display the measurement result in one candidate region having a best score.

An information processing method according to the aspect of the present disclosure comprises:
via a computer,
acquiring a measurement result of an object included in a medical image;
recognizing an organ region including the object in the medical image;
deriving a score representing suitability for displaying the measurement result based on a ratio at which a measurement label, which includes the measurement result, and the organ region overlap each other, for each of a plurality of candidate regions for displaying the measurement result around the object; and
displaying the measurement result in at least one candidate region in which the score satisfies a criterion.

An information processing program according to the aspect of the present disclosure causes a computer to execute: a procedure of acquiring a measurement result of an object included in a medical image;
a procedure of recognizing an organ region including the object in the medical image;
a procedure of deriving a score representing suitability for displaying the measurement result based on a ratio at which a measurement label, which includes the measurement result, and the organ region overlap each other, for each of a plurality of candidate regions for displaying the measurement result around the object; and
a procedure of displaying the measurement result in at least one candidate region in which the score satisfies a criterion.

It should be noted that the technology of the present disclosure may be applied to a program product.

According to the present disclosure, it is possible to display a measurement result of an object included in a medical image at a position that does not interfere with image interpretation.

### BRIEF DESCRIPTION OF THE DRAWINGS

FIG. 1 is a diagram showing a schematic configuration of an information processing system to which an information processing apparatus according to an embodiment of the present disclosure is applied.

FIG. 2 is a diagram showing a hardware configuration of the information processing apparatus according to the present embodiment.

FIG. 3 is a functional configuration diagram of the information processing apparatus according to the present embodiment.

FIG. 4 is a diagram showing a lung field region and a lesion extracted from a medical image.

FIG. 5 is a diagram for describing measurement of a size of the lesion.

FIG. 6 is a diagram for describing a plurality of candidate regions.

FIG. 7 is a diagram showing a measurement label.

FIG. 8 is a diagram showing a state in which a measurement label is disposed in a candidate region related to a first control point.

FIG. 9 is a diagram showing a state in which a measurement label is disposed in a candidate region related to a second control point.

FIG. 10 is a diagram for describing derivation of a score in which an element other than a ratio of the measurement label overlapping with the lung field region is taken into consideration.

FIG. 11 is a diagram for describing derivation of a score in which an element other than a ratio of the measurement label overlapping with the lung field region is taken into consideration.

FIG. 12 is a diagram for describing derivation of a score in which an element other than a ratio of the measurement label overlapping with the lung field region is taken into consideration.

FIG. 13 is a diagram for describing derivation of a score in which an element other than a ratio of the measurement label overlapping with the lung field region is taken into consideration.

FIG. 14 is a diagram showing a medical image on which a measurement result is displayed.

FIG. 15 is a flowchart showing processing performed in the present embodiment.

### DETAILED DESCRIPTION

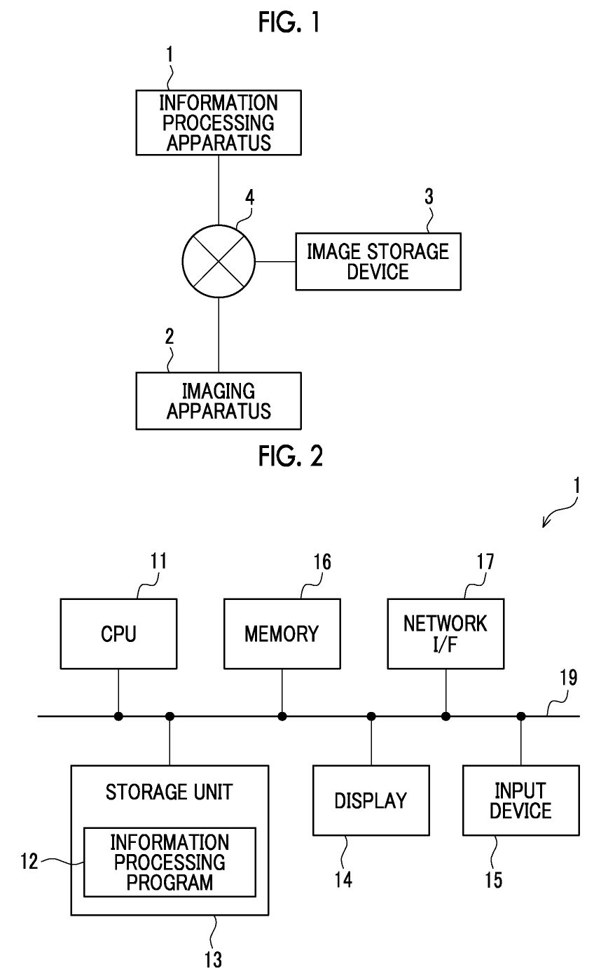

Hereinafter, embodiments of the present disclosure will be described with reference to the drawings. First, a configuration of an information processing system to which an information processing apparatus according to the present embodiment is applied will be described. FIG. 1 is a diagram showing a schematic configuration of the information processing system. In the information processing system shown in FIG. 1, an information processing apparatus 1 that encompasses the information processing apparatus according to the present embodiment, an imaging apparatus 2, and an image storage device 3 are connected to each other via a wired or wireless network 4 in a communicable state. The network 4 is, for example, a network such as a local area network (LAN) and a wide area network (WAN).

The information processing apparatus 1 is a computer on which an information processing program according to the present embodiment is installed. The computer may be a workstation or a personal computer directly operated by a doctor performing diagnosis, or may be a server computer connected to a workstation and to a personal computer via a network. The information processing program is stored in a storage device of the server computer connected to the network or in a network storage in a state of being accessible from the outside, and is downloaded and installed in the computer used by a doctor in response to a request. Alternatively, the information processing program is distributed by being recorded on a recording medium such as a digital versatile disc (DVD) or a compact disc read only memory (CD-ROM), and is installed on the computer from the recording medium.

The imaging apparatus 2 is an apparatus that generates a medical image representing a site of a subject that is a diagnosis target by imaging the site, and specifically, the imaging apparatus 2 is an apparatus that acquires a three-dimensional image, such as a CT apparatus, an MRI apparatus, or a positron emission tomography (PET) apparatus, or an apparatus that acquires a two-dimensional image, such as a radiography apparatus. The medical image generated by the imaging apparatus 2 is transmitted to the image storage device 3 and is stored.

The image storage device 3 is a computer that stores and manages various types of data, and comprises a large-capacity external storage device and database management software. The image storage device 3 communicates with other apparatuses via a wired or wireless network 4, and transmits and receives image data of the medical image and the like. Specifically, various types of data including the image data of the medical image generated by the imaging apparatus 2 are acquired via the network and stored in a recording medium such as a large-capacity external storage device and managed. The storage format of the image data and the communication between the respective apparatuses via the network 4 are based on a protocol such as digital imaging and communication in medicine (DICOM).

Next, the information processing apparatus according to the present embodiment will be described. FIG. 2 is a diagram showing a hardware configuration of the information processing apparatus according to the present embodiment. As shown in FIG. 2, the information processing apparatus 1 includes a central processing unit (CPU) 11, a memory 16 as a temporary storage area, and a non-volatile storage unit 13. In addition, the information processing apparatus 1 includes a display 14 such as a liquid crystal display or an organic electro-luminescence (EL) display, and an input device 15 such as a keyboard. The display 14 and the input device 15 may be integrated as a touch panel display. In addition, the information processing apparatus 1 includes a network interface (I/F) 17 connected to the network 4. The CPU 11, the memory 16, the storage unit 13, and the network I/F 17 are connected to a bus 19.

The storage unit 13 is implemented by a hard disk drive (HDD), a solid state drive (SSD), a flash memory, or the like. An information processing program 12 is stored in the storage unit 13 as a storage medium. The CPU 11 reads out the information processing program 12 from the storage unit 13, develops the information processing program 12 into the memory 16, and executes the developed information processing program 12.

The information processing program 12 is stored in a storage device of the server computer connected to the network or in a network storage in a state of being accessible from the outside, and is downloaded and installed on the information processing apparatus 1 in response to a request.

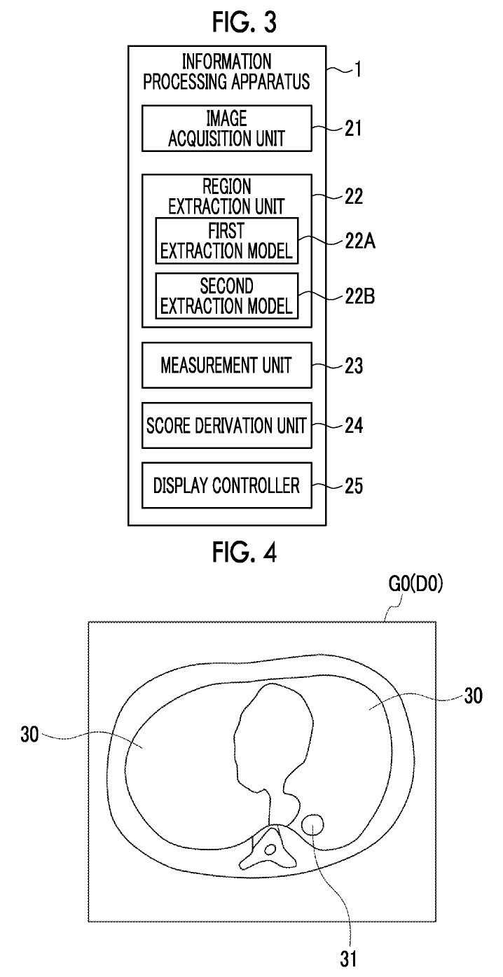

Next, a functional configuration of the information processing apparatus according to the present embodiment will be described. FIG. 3 is a diagram showing the functional configuration of the information processing apparatus according to the present embodiment. As shown in FIG. 3, the information processing apparatus 1 comprises an image acquisition unit 21, a region extraction unit 22, a measurement unit 23, a score derivation unit 24, and a display controller 25. Then, the CPU 11 executes the information processing program 12, thereby the CPU 11 functions as the image acquisition unit 21, the region extraction unit 22, the measurement unit 23, the score derivation unit 24, and the display controller 25.

The image acquisition unit 21 acquires a medical image G0 to be diagnosed from the imaging apparatus 2 and/or the image storage device 3. In the present embodiment, the medical image G0 is a CT image including a chest of a patient.

The region extraction unit 22 extracts a region of an object from the medical image G0. The object includes an organ and a lesion included in the organ. Therefore, the region extraction unit 22 has a first extraction model 22A that has been trained through machine learning to extract a target organ from the medical image G0, and extracts the target organ using the first extraction model 22A. In addition, the region extraction unit 22 has a second extraction model 22B that extracts a region of a lesion from the medical image G0, and extracts the lesion from the medical image G0 using the second extraction model 22B. The second extraction model 22B may extract a lesion from the organ extracted from the medical image G0 by the first extraction model 22A. The region extraction unit 22 may extract an organ and a lesion using threshold value processing, template matching, or the like instead of the first extraction model 22A and the second extraction model 22B.

In the present embodiment, the region extraction unit 22 extracts a lung field region, which is one of the organs included in the medical image G0, and extracts a lesion such as a nodule included in the lung field region as an object. FIG. 4 is a diagram showing a lung field region and a lesion extracted from the medical image G0. As shown in FIG. 4, a lung field region 30 and a lesion 31 are extracted in a tomographic image D0 of a certain tomographic plane included in the medical image G0.

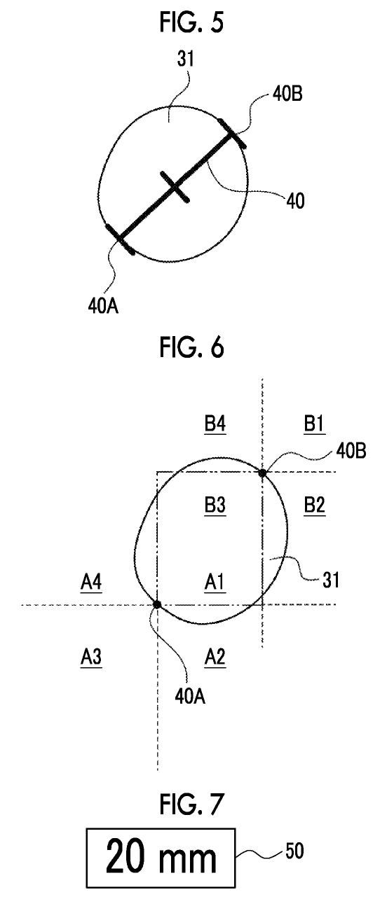

The measurement unit 23 derives a measurement result by measuring a size, a signal value, and the like of the lesion extracted from the medical image G0 by the region extraction unit 22. In the present embodiment, the measurement unit 23 measures the size of the lesion. FIG. 5 is a diagram for describing the measurement of the size of the lesion. In FIG. 5, the lesion 31 included in the tomographic image D0 shown in FIG. 4 is shown in an enlarged manner. In a case of measuring the size of the lesion 31, the measurement unit 23 displays a measurement object 40 on the tomographic image D0. The measurement object 40 has an I-shape having a first control point 40A and a second control point 40B.

The operator performs an operation of matching the first control point 40A of the measurement object 40 with a lower left corner of the lesion 31 by using the input device 15, and then performs an operation of matching a length of the measurement object 40 with a diameter of the lesion 31 by moving the second control point 40B of the measurement object 40 to an upper right corner of the lesion 31. The measurement unit 23 counts the number of voxels of the measurement object 40 by receiving the operation by the operator. Then, the measurement unit 23 derives a diameter of the lesion 31 in a direction along the measurement object 40 as a measurement result by multiplying a size per voxel of the medical image G0 by the number of voxels of the measurement object 40.

The score derivation unit 24 derives a ratio at which a measurement label including the measurement result and the extracted organ region overlap each other for each of a plurality of candidate regions for displaying the measurement result around the lesion that is the object. Then, the score derivation unit 24 derives a score representing suitability for displaying the measurement result, based on the derived ratio. In the present embodiment, the measurement is performed using the measurement object 40 having the two control points, which are the first control point 40A and the second control point 40B, as described above. Therefore, the score derivation unit 24 sets the plurality of candidate regions for displaying the measurement result around each of the first control point 40A and the second control point 40B of the measurement object 40.

FIG. 6 is a diagram for describing the plurality of candidate regions. FIG. 6 shows a candidate region for the lesion 31. In addition, the display of the measurement object 40 is omitted, and only the first control point 40A and the second control point 40B are shown. The score derivation unit 24 sets candidate regions A1 to A4 in each of an upper right side, a lower right side, a lower left side, and an upper left side of the first control point 40A by dividing a region around the first control point 40A into four equal parts with respect to the first control point 40A. In addition, the score derivation unit 24 sets candidate regions B1 to B4 in each of an upper right side, a lower right side, a lower left side, and an upper left side of the second control point 40B by dividing a region around the second control point 40B into four equal parts with respect to the second control point 40B.

The score derivation unit 24 derives a ratio at which the measurement label and the organ region overlap each other for each of the plurality of candidate regions by disposing the measurement label including the measurement result in the plurality of candidate regions. FIG. 7 is a diagram showing the measurement label. As shown in FIG. 7, a measurement label 50 has a rectangular region having a size surrounding a numerical value (20 mm in FIG. 7) representing the measurement result. FIG. 7 shows the measurement label 50 including the size of the lesion 31, which is "20 mm", as the measurement result. However, in a case where the measurement result includes a signal value or the like in addition to the size, since the measurement result extends over a plurality of lines, the size of the measurement label 50 also increases. A character size of the measurement result included in the measurement label 50 is set to an appropriate size in advance.

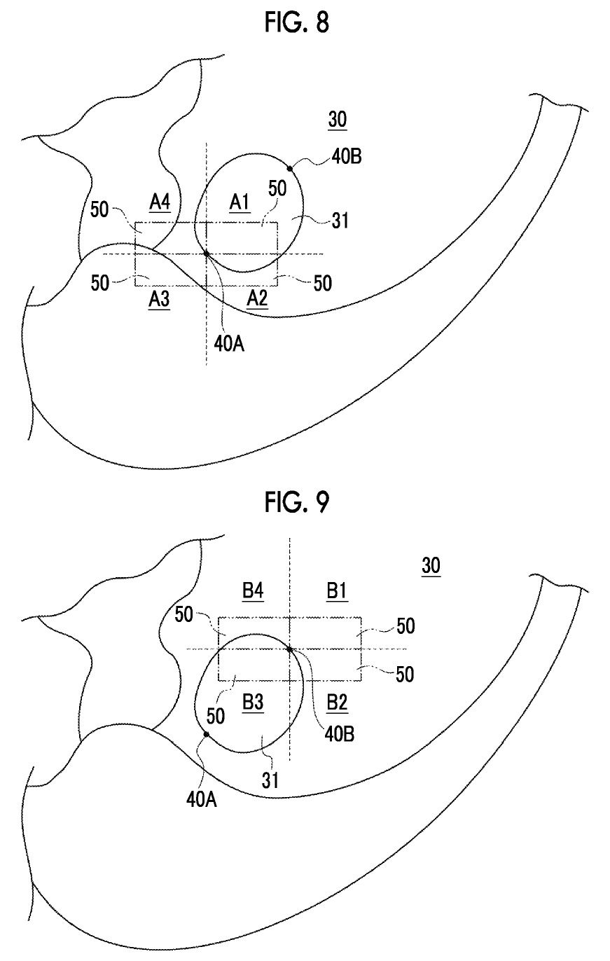

First, the score derivation unit 24 disposes the measurement label 50 in the candidate regions A1 to A4 related to the first control point 40A. FIG. 8 is a diagram showing a state in which the measurement label is disposed in the candidate region related to the first control point 40A. The score derivation unit 24 derives a ratio at which the measurement label 50 disposed in each of the candidate regions A1 to A4 overlaps the lung field region 30, which is the organ region including the lesion 31, by a percentage. For example, in the candidate regions A1 and A2, since the entire region of the measurement label 50 overlaps the lung field region 30, the ratio of overlap is 100%. In the candidate region A3, since 40% of the measurement label 50 overlaps the lung field region 30, the ratio of overlap is 40%. In the candidate region A4, since 60% of the measurement label 50 overlaps the lung field region 30, the ratio of overlap is 60%. The score derivation unit 24 derives a score representing the suitability for displaying the measurement result by setting 0 as a reference value and subtracting the ratio of overlap from the reference value for each of the candidate regions A1 to A4 of the first control point 40A. In the present embodiment, the scores of the candidate regions A1 to A4 of the first control point 40A are -100, -100, -40, and -60.

Similarly, the score derivation unit 24 disposes the measurement label 50 in the candidate regions B1 to B4 related to the second control point 40B, and derives a ratio at which the measurement label 50 overlaps the lung field region 30 by a percentage for each of the measurement labels 50. FIG. 9 is a diagram showing a state in which the measurement label is disposed in the candidate region related to the second control point 40B. As shown in FIG. 9, since the entire region of the measurement label 50 overlaps the lung field region 30 in all of the candidate regions B1 to B4, the ratio of overlap is 100% in all of the candidate regions B1 to B4. Therefore, the scores of the candidate regions B1 to B4 of the second control point 40B are all -100.

The score derivation unit 24 determines the candidate region having the best score among the plurality of candidate regions as the region in which the measurement result is to be displayed. In the present embodiment, as the score increases, the ratio at which the measurement label 50 overlaps the lung field region 30 decreases, and thus the suitability for displaying the measurement result increases. Therefore, the score derivation unit 24 determines the candidate region having the maximum score as the region in which the measurement result is to be displayed. In the present embodiment, the candidate region A3 for the first control point 40A having the maximum score of -40 is determined as the region in which the measurement result is to be displayed.

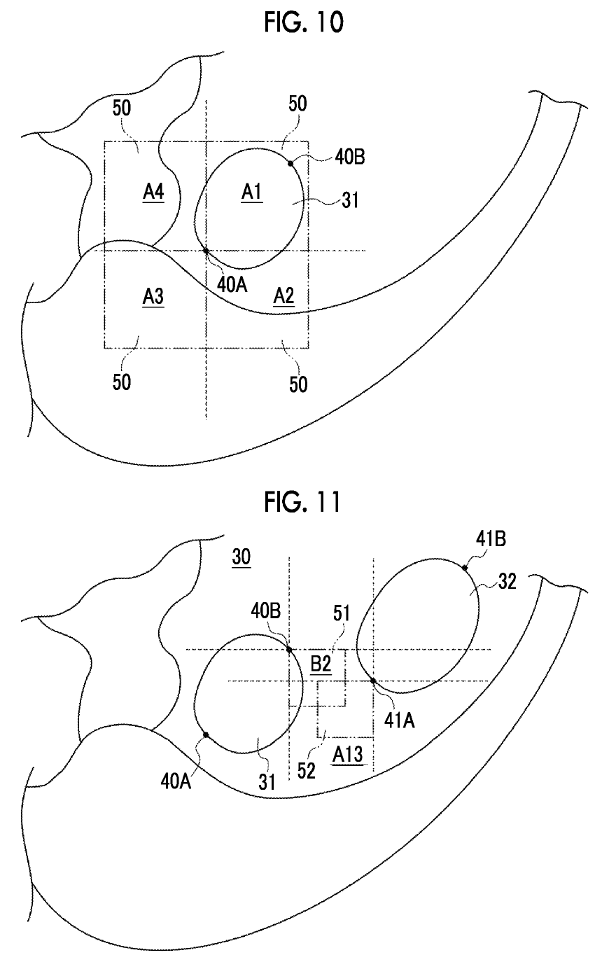

In a case of deriving the score, an element other than the ratio at which the measurement label 50 overlaps the lung field region 30 may be taken into consideration. For example, in a case where the number of items of the measurement result is large and the size of the measurement label 50 is large as shown in FIG. 10, the candidate region A1 related to the first control point 40A overlaps the second control point 40B. In such a case, the measurement result is difficult to see in a case where the measurement result is displayed in the candidate region A1. Therefore, the score derivation unit 24 may reduce the score for the candidate region in which the disposed measurement label 50 overlaps the second control point 40B among the candidate regions A1 to A4 related to the first control point 40A, so that the suitability is reduced. For example, the score may be further set to -100. In this case, the first control point 40A is an example of one control point of the present disclosure, and the second control point 40B is an example of another control point of the present disclosure.

In addition, in the present embodiment, it is assumed that a second lesion 32 is extracted in addition to the lesion 31. A candidate region for displaying the measurement label is determined for the lesion 32 in the same manner as for the lesion 31. In this case, as shown in FIG. 11, it is assumed that a measurement label 51 is disposed in a candidate region B2 related to the second control point 40B for the lesion 31, and a measurement label 52 is disposed in a candidate region A13 related to a first control point 41A for the lesion 32. In this case, the disposed measurement label 51 and the measurement label 52 overlap each other. In such a case, the score derived for the candidate region B2 and the candidate region A13 may be further reduced so that the suitability is reduced. For example, the score may be further set to -100.

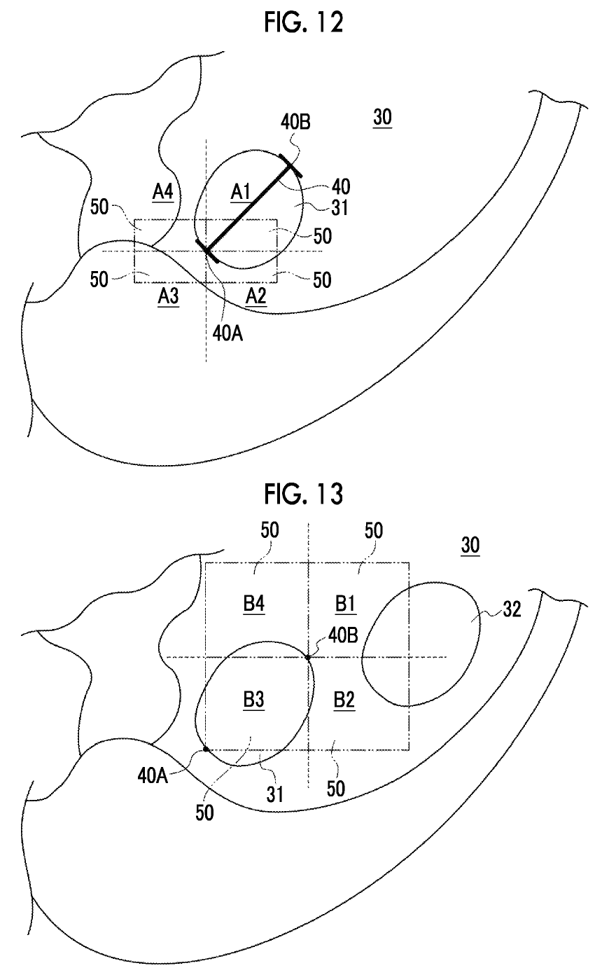

In addition, as shown in FIG. 12, the measurement label 50 disposed in the candidate region A1 related to the first control point 40A overlaps the measurement object 40 of the lesion 31. In this case, the score of the candidate region A1 may be further subtracted so that the suitability is reduced. For example, the score may be further set to -100.

In addition, as shown in FIG. 13, in a case where the measurement label 50 disposed in the candidate regions B1 and B2 related to the second control point 40B of the lesion 31 overlaps the lesion 32, the score derived for the candidate regions B1 and B2 may be further subtracted so that the suitability is reduced. For example, the score may be further set to -100.

In a case where the measurement label 50 overlaps the measurement object 40 as shown in FIG. 12 and in a case where the measurement label 50 overlaps the other lesion 32 as shown in FIG. 13, more values may be reduced from the score than in the cases shown in FIGS. 10 and 11. For example, the score may be set to -200. As described above, by changing the degree of the score reduction, the measurement result can be displayed at a more appropriate position.

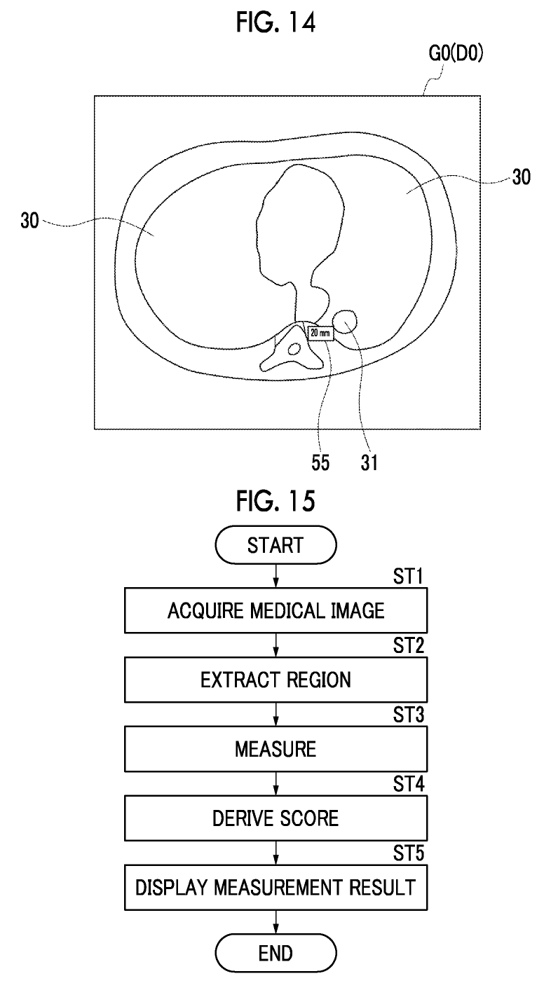

The display controller 25 displays the measurement result in the determined candidate region, that is, the candidate region having the best score. FIG. 14 is a diagram showing a medical image in which the measurement result is displayed. FIG. 14 shows a tomographic image D0 of one tomographic plane included in a medical image G0. In the present embodiment, the candidate region A3 for the first control point 40A is determined as a region in which the measurement result is displayed. Therefore, the display controller 25 displays a measurement result 55 of the lesion 31 in a lower left region of the lesion 31 included in the medical image G0 (tomographic image D0).

The measurement result 55 is displayed in a mode different from a mode of the candidate region to be disposed. For example, in the present embodiment, the medical image G0 is a CT image. The CT image is a monochrome image. Therefore, the measurement result 55 may be displayed in a color having a relatively high chroma saturation, for example, light pink, yellow-green, or yellow. In addition, the measurement result 55 may be displayed in a mode different from the mode of the candidate region to be disposed by surrounding the measurement result 55 with a rectangular region to which a color is assigned.

Then, processing performed in the present embodiment will be described. FIG. 15 is a flowchart showing the processing performed in the present embodiment. First, the image acquisition unit 21 acquires the medical image G0 (step ST1). The region extraction unit 22 extracts regions of the organ and the object from the medical image G0 (step ST2). The measurement unit 23 measures a size, a signal value, and the like of the lesion extracted from the medical image G0 (step ST3).

The score derivation unit 24 derives a score representing suitability for displaying the measurement result (step ST4). The display controller 25 displays the measurement result in the candidate region having the maximum score (step ST5), and ends the processing.

As described above, in the present embodiment, for each of the plurality of candidate regions for displaying the measurement result around the object such as the lesion, the score representing the suitability for displaying the measurement result is derived based on the ratio at which the measurement label 50 including the measurement result and the organ region overlap each other, and the measurement result is displayed in the candidate region having the best score. Therefore, even in a case where the measurement result overlaps the organ or the object on which the measurement is performed, the measurement result can be displayed at a position where the overlap is as small as possible. Therefore, the measurement result of the lesion included in the medical image G0 can be displayed at a position that does not interfere with the interpretation of the medical image G0.

In the above-described embodiment, since the score derivation unit 24 derives the score by the reduction method, the candidate region having the maximum score is derived as the best candidate region, but the method of deriving the score is not limited thereto. For example, the ratio at which the measurement label including the measurement result and the extracted organ region overlap each other may be used as the score. In this case, the candidate region having the minimum score is the best candidate region. In addition, in this case, in a case where an element other than the ratio at which the measurement label 50 overlaps the lung field region 30 is taken into consideration, the score is reduced by adding the score.

In the above-described embodiment, the measurement of the object included in the medical image G0 is performed in the information processing apparatus 1 according to the present embodiment, but the present disclosure is not limited thereto. The measurement result measured in another apparatus may be stored in the image storage device 3 together with the medical image G0, and in a case where the medical image G0 is acquired from the image storage device 3 in the information processing apparatus 1 according to the present embodiment, the measurement result may be acquired together.

In addition, in the above-described embodiment, the measurement result is displayed in the candidate region having the best score among the plurality of candidate regions, but the present disclosure is not limited thereto. The measurement result may be displayed in at least one candidate region in which the score satisfies a criterion. In this case, for example, the candidate region in which the score is equal to or greater than a predetermined threshold value may be used as the candidate region satisfying the criterion. In a case where there are a plurality of candidate regions in which the score satisfies the criterion, the measurement result may be displayed in any one of the candidate regions, or the measurement result may be displayed in all the candidate regions satisfying the criterion. For example, in a case where the scores of the candidate regions at the upper right and lower right of the control point satisfy the criterion, the measurement result may be displayed in the two candidate regions at the upper right and lower right of the control point.

In the present embodiment, each type of process is executed by any computer. In addition, any computer may execute these processes using a processor as hardware, a program as software, or a combination thereof. In that case, the processor is configured to execute various types of processes in the present embodiment in cooperation with the program, and may function as each unit or each means in the present embodiment. Further, the execution order of the processes by the processor is not limited to the order described and may be changed as appropriate. Any computer may be a general purpose computer, a special purpose computer, a workstation, or other system capable of executing each process.

The processor may be configured with one or a plurality of pieces of hardware, and the type of hardware is not limited. For example, the processor may be configured with hardware, such as a central processing unit (CPU), a micro processing unit (MPU), a programmable logic device, such as a field programmable gate array (FPGA), a dedicated circuit that is used to execute specific processing, such as an application-specific integrated circuit (ASIC), a graphic processing unit (GPU), or a neural processing unit (NPU). Additionally, the type of hardware may be a combination of different types of hardware. In a case where a plurality of hardware components are configured to execute one or a plurality of processes of a certain processor, the plurality of hardware components may be present in devices physically separate from each other or may be present within the same device. Further, in any of the embodiments, the order of each process performed by the processor is not limited to the order described above, and may be changed as appropriate. The hardware is configured by an electrical circuit (circuitry) in which circuit elements such as semiconductor elements are combined.

Further, the program may be software, such as firmware or a microcode. In addition, the program may be, for example, a program module group, and each function thereof may be implemented by a processor configured to execute each function. The program may be a program code or a plurality of code segments stored in one or a plurality of non-transitory computer-readable media (for example, storage media, other storages, or the like). The program may be divided and stored in the plurality of non-transitory computer-readable media present in devices physically separate from each other. The program code or code segment may represent any combination of a procedure, a function, a subprogram, a routine, a subroutine, a module, a software package, a class, or an instruction, a data structure, or a program statement. The program code or code segment may be connected to another code segment or a hardware circuit by transmitting and receiving information, data, an argument, a parameter, or a content of a memory.

Further, in the above-described embodiment, the aspect has been described in which the information processing program 12 is stored (installed) in the storage unit 13 in advance, but the present disclosure is not limited thereto. The information processing program 12 may be provided in a form recorded on a recording medium, such as a compact disc read-only memory (CD-ROM), a digital versatile disc read-only memory (DVD-ROM), and a universal serial bus (USB) memory. Alternatively, the information processing program 12 may be provided in a form that can be downloaded from an external device via a network.

The technology of the present disclosure extends to all program products. The program product includes products in any aspect for providing the program. For example, the program product includes a program provided through a network such as the Internet, a non-transitory computer-readable recording medium, such as a CD-ROM, a DVD, and a USB memory in which the program is stored, and the like.

The supplementary notes of the present disclosure will be described below.

### Supplementary Note 1

An information processing apparatus comprising:
a processor,
in which the processor is configured to:
   acquire a measurement result of an object included in a medical image;
   recognize an organ region including the object in the medical image;
   derive a score representing suitability for displaying the measurement result based on a ratio at which a measurement label, which includes the measurement result, and the organ region overlap each other, for each of a plurality of candidate regions for displaying the measurement result around the object; and
   display the measurement result in a candidate region having a best score.

### Supplementary Note 2

The information processing apparatus according to Supplementary Note 1, in which the processor is configured to:
derive the ratio for each of the plurality of candidate regions by disposing the measurement label in the plurality of candidate regions; and
derive the score for each of the plurality of candidate regions based on the ratio.

### Supplementary Note 3

The information processing apparatus according to Supplementary Note 2, in which the processor is configured to:
display a measurement object including at least two control points for measurement on the object of the medical image;
acquire the measurement result by receiving an operation on the control point of the measurement object;
set the plurality of candidate regions based on each of the at least two control points of the measurement object; and
reduce the score in a case where the measurement label disposed in each of a plurality of candidate regions based on one control point of the at least two control points (i) overlaps with another control point other than the one control point, (ii) overlaps with the measurement label disposed in each of the plurality of candidate regions based on the other control point, or (iii) overlaps with the measurement object including the one control point.

### Supplementary Note 4

The information processing apparatus according to Supplementary Note 2 or 3, in which the processor is configured to reduce the score in a case where each of the plurality of candidate regions for one object overlaps with another object in a case of displaying the measurement result of a plurality of the objects.

### Supplementary Note 5

The information processing apparatus according to Supplementary Note 2, in which the processor is configured to:
display a measurement object including at least two control points for measurement on the object of the medical image;
acquire the measurement result by receiving an operation on the control point of the measurement object;
set the plurality of candidate regions based on each of the at least two control points of the measurement object;
reduce the score in a case where the measurement label disposed in each of a plurality of candidate regions based on one control point of the at least two control points (i) overlaps with another control point other than the one control point, (ii) overlaps with the measurement label disposed in each of the plurality of candidate regions based on the other control point, or (iii) overlaps with the measurement object including the one control point;
further reduce the score (iv) in a case where each of the plurality of candidate regions for one object overlaps with another object in a case of displaying the measurement result of a plurality of the objects; and
increase a degree of the score reduction in a case of (iii) and (iv) than in a case of (i) and (ii).

### Supplementary Note 6

The information processing apparatus according to any one of Supplementary Notes 1 to 5,
in which the plurality of candidate regions include four regions of upper right, lower right, lower left, and upper left of at least two control points included in a measurement object for acquiring the measurement result.

### Supplementary Note 7

The information processing apparatus according to any one of Supplementary Notes 1 to 6,
in which the processor is configured to display the measurement result in a mode different from a mode of the candidate region for displaying the measurement result.

### Supplementary Note 8

The information processing apparatus according to any one of Supplementary Notes 1 to 7,
in which the processor is configured to display the measurement result in one candidate region having a best score.

### Supplementary Note 9

An information processing method comprising:
via a computer,
acquiring a measurement result of an object included in a medical image;
recognizing an organ region including the object in the medical image;
deriving a score representing suitability for displaying the measurement result based on a ratio at which a measurement label, which includes the measurement result, and the organ region overlap each other, for each of a plurality of candidate regions for displaying the measurement result around the object; and
displaying the measurement result in a candidate region having a best score.

### Supplementary Note 10

An information processing program causing a computer to execute:
a procedure of acquiring a measurement result of an object included in a medical image;
a procedure of recognizing an organ region including the object in the medical image;
a procedure of deriving a score representing suitability for displaying the measurement result based on a ratio at which a measurement label, which includes the measurement result, and the organ region overlap each other, for each of a plurality of candidate regions for displaying the measurement result around the object; and
a procedure of displaying the measurement result in a candidate region having a best score.

What is claimed is:

1. An information processing apparatus comprising:
a processor,
wherein the processor is configured to:
acquire a measurement result of an object included in a medical image;
recognize an organ region including the object in the medical image;
derive a score representing suitability for displaying the measurement result based on a ratio at which a measurement label, which includes the measurement result, and the organ region overlap each other, for each of a plurality of candidate regions for displaying the measurement result around the object; and
display the measurement result in at least one candidate region in which the score satisfies a criterion.

2. The information processing apparatus according to claim 1,
wherein the processor is configured to:
derive the ratio for each of the plurality of candidate regions by disposing the measurement label in the plurality of candidate regions; and
derive the score for each of the plurality of candidate regions based on the ratio.

3. The information processing apparatus according to claim 2,
wherein the processor is configured to:
display a measurement object including at least two control points for measurement on the object of the medical image;
acquire the measurement result by receiving an operation on the control point of the measurement object;
set the plurality of candidate regions based on each of the at least two control points of the measurement object; and
reduce the score in a case where the measurement label disposed in each of a plurality of candidate regions based on one control point of the at least two control points (i) overlaps with another control point other than the one control point, (ii) overlaps with the measurement label disposed in each of the plurality of candidate regions based on the other control point, or (iii) overlaps with the measurement object including the one control point.

4. The information processing apparatus according to claim 2,
wherein the processor is configured to reduce the score in a case where each of the plurality of candidate regions for one object overlaps with another object in a case of displaying the measurement result of a plurality of the objects.

5. The information processing apparatus according to claim 2,
wherein the processor is configured to:
display a measurement object including at least two control points for measurement on the object of the medical image;
acquire the measurement result by receiving an operation on the control point of the measurement object;
set the plurality of candidate regions based on each of the at least two control points of the measurement object;
reduce the score in a case where the measurement label disposed in each of a plurality of candidate regions based on one control point of the at least two control points (i) overlaps with another control point other than the one control point, (ii) overlaps with the measurement label disposed in each of the plurality of candidate regions based on the other control point, or (iii) overlaps with the measurement object including the one control point;
further reduce the score (iv) in a case where each of the plurality of candidate regions for one object overlaps with another object in a case of displaying the measurement result of a plurality of the objects; and
increase a degree of the score reduction in a case of (iii) and (iv) than in a case of (i) and (ii).

6. The information processing apparatus according to claim 1,
wherein the plurality of candidate regions include four regions of upper right, lower right, lower left, and upper left of at least two control points included in a measurement object for acquiring the measurement result.

7. The information processing apparatus according to claim 1,
wherein the processor is configured to display the measurement result in a mode different from a mode of the candidate region for displaying the measurement result.

8. The information processing apparatus according to claim 1,
wherein the processor is configured to display the measurement result in one candidate region having a best score.

9. An information processing method comprising:
via a computer,
acquiring a measurement result of an object included in a medical image;
recognizing an organ region including the object in the medical image;
deriving a score representing suitability for displaying the measurement result based on a ratio at which a measurement label, which includes the measurement result, and the organ region overlap each other, for each of a plurality of candidate regions for displaying the measurement result around the object; and
displaying the measurement result in at least one candidate region in which the score satisfies a criterion.

10. A non-transitory computer-readable storage medium that stores an information processing program causing a computer to execute:
a procedure of acquiring a measurement result of an object included in a medical image;
a procedure of recognizing an organ region including the object in the medical image;
a procedure of deriving a score representing suitability for displaying the measurement result based on a ratio at which a measurement label, which includes the measurement result, and the organ region overlap each other, for each of a plurality of candidate regions for displaying the measurement result around the object; and
a procedure of displaying the measurement result in at least one candidate region in which the score satisfies a criterion.
